(19) United States
(12) Patent Application Publication
(10) Pub. No.: US 2021/0134437 A1
(43) Pub. Date: May 6, 2021

(54) COMPUTER IMPLEMENTED METHOD AND MODULE FOR ACCESSING A DICOM WHOLE SLIDE IMAGE DATABASE

(71) Applicant: Servicio Andaluz De Salud, Sevilla (ES)

(72) Inventors: David DE MENA GARCIA, Sevilla (ES); Alfonso SANCHEZ GARCIA, Sevilla (ES); Marcial GARCIA ROJO, Sevilla (ES)

(21) Appl. No.: 17/256,970

(22) PCT Filed: Jun. 28, 2019
(86) PCT No.: PCT/EP2019/067360

§ 371 Date: Dec. 29, 2020

(30) Foreign Application Priority Data
Jun. 29, 2018 (ES) P201830651

(51) Int. Cl. G16H 30/20; G16H 30/40; G16H 50/70; G06F 16/51

(52) U.S. Cl. CPC G16H 30/20; G06F 16/51; G16H 50/70; G16H 30/40

(57) ABSTRACT

The invention discloses a computer implemented method capable of “translating” a request from a proprietary high-resolution image viewer made in terms of level, row, and column into a DICOM valid request made in terms of DICOM instance and DICOM frame number.

### OBJECT OF THE INVENTION

The object of the present invention is a new computer implemented method and module for displaying a DICOM Whole Slide Image (WSI), where each frame is identified by instance and frame number, using a conventional high-resolution image viewer where each cell is identified by level, row and column.

### BACKGROUND ART

### Digitization Technology

In the field of medicine, particularly in Anatomic Pathology, the need to make histological and cytological slides for a subsequent microscopic study by a pathologist is known. A number of devices for a complete digitization of those slides have recently appeared. Digitization devices, known as scanners, obtain a high-resolution digital image of the slide, which is thus available for analysis any place and time without the need for the clinician to have physical access to the slide.

The digitization method carried out by these scanners for obtaining a digital high-resolution image of the slides comprises taking a mosaic of photographs, normally square-shaped photographs, of small fragments of the surface of the slide known as “cells”. The scanners comprise a digital camera with a 20× to 40× zoom lens, mounted on a structure holding the camera over the slide. A parallel relative movement between the camera and the slide is generated while the camera takes multiple pictures of the slide from above. All those pictures are taken in positions separated by a small distance, such as microns. Subsequently, every photograph is placed in the correct position in the image. FIG. 1 shows an exemplary image of a digitized histological slide according with this method.

A drawback of this technology is related with the huge size of the complete digitized high-resolution image. Indeed, a high-resolution image of this type may contain several thousands of individual photographs. The size of such a high-resolution image may therefore range between several hundreds of megabytes to about tens of gigabytes. Obviously, managing and displaying an image having this size is very complicated.

In order to solve this drawback, the use of image composition method known as “pyramidal” for displaying these high-resolution images is known. This method comprises creating a number of lower resolution images usually known as “levels”. Usually, the size of the image making up each level is half the size of the image making up the level immediately below. The image of each level is also formed by a plurality of cells. Therefore, starting from the complete image having the highest resolution that makes up the lowermost level at the base of the pyramid, successive levels having gradually lower resolutions are created, until the uppermost level having the lowest resolution that makes up the top of the pyramid. FIG. 2a schematically shows the pyramidal structure in a case where the original image is level 0 of the pyramid and the top of the pyramid is level 3, where each level corresponds to a particular reduction factor with respect to the complete image. On the other hand, FIG. 2b shows the image corresponding to each level making up the pyramid of FIG. 2a.

When displaying the image, initially the uppermost level having the lowest resolution is displayed (in the example shown in FIGS. 2a and 2b, level 3). When the user desires to view a particular area of the image with a greater resolution, the immediately lower level is accessed. This process takes place as many times as necessary until the highest resolution image at the base of the pyramid is displayed (in the present example, successively level 2, level 1 and level 0). This method effectively limits the size of the image files managed by a particular viewer software at any given moment. Indeed, the viewer requests a database storing all the image data only the data in connection with the relevant cells at a given moment. This image visualization method is very similar to the method employed Google Maps®, a tool provided by Google®.

Therefore, the current digitalization technology mainly provides three elements for defining an image: image, level, and cell. Each image comprises several levels, and every level in turn comprises a plurality of cells each corresponding to an individual photograph. Each level is half the size the level below, and an image contains levels corresponding to all integer reduction factors until arriving at a single-cell uppermost level. In this context, each particular cell in a particular image is univocally identified by means of three parameters: level, row and column.

FIG. 3 schematically shows a particular image having 4 levels where each level is half the size of the level immediately below: level 0 is an 8×8 frame image (reduction factor 0), level 1 is a 4×4 frame image (reduction factor 1), level 2 is a 2×2 frame image (reduction factor 2), and level 3 contains a single frame (reduction factor 3). According to the structure disclosed above, each of the cells in each level of the image is identified by means of three parameters: level, row, and column. Therefore, exemplary cells A and B would be identified as follows:
Cell A: Level 0, Row 2, Column 4.
Cell B: Level 1, Row 3, Column 2

A dedicated system stores the above-defined cell position information along with additional relevant information the information. The information may include, for example, number and/or size of the levels, image files containing the photograph corresponding to each cell (e.g. a jpg file), etc. During navigation throughout the image, known proprietary viewers communicate with the system for requesting the information needed to display the image with the resolution required by the user. In particular, these proprietary viewers request a relevant cell image file by identifying the level, row and column of the necessary cell. In order to function properly, therefore, current proprietary viewers require the relevant image database to store the image information according to the above structure.

DICOM standard

DICOM (Digital Imaging and COmmunication in Medicine) is a standard for storing and exchanging medical images enabling the integration of medical imaging devices such as scanners, servers, workstations, printers, network hardware, and picture archiving and communication systems (PACS) from multiple manufacturers. It has been widely adopted by hospitals. Further, supplements 145 and 122 of DICOM define the necessary extensions for storing and visualizing high-resolution images used in Anatomic Pathology.

A simplified entity-relationship defined by DICOM establishes the “patient” as the main node. Each patient may have a number of “studies”. Each study may be divided in one or more “series”. Each series may comprise several “instances”. And, finally, each instance may comprise a single image or an image in a plurality of “frames”. For Digital Pathology images, DICOM Supplement 145 defines the use of multiframe images. This structure corresponds to a class denoted as VL Whole Slide Microscopy Image, (WSI).

So the DICOM standard, for a given patient and study, defines three elements essentially equivalent to those disclosed by the scanner manufacturers for every WSI: series, instance, and frame. Every series comprises several instances and, in turn, every instance can comprise a plurality of frames. However, unlike existing conventional proprietary viewers, the DICOM standard only allows access to a particular frame inside an instance by means of the number of frame. Further, DICOM supplement 145 is not strict as to the order of the frame numbers, and therefore frame numbers can be arbitrarily assigned to each frame in an instance. Furthermore, the DICOM standard is not strict also in identification of instances in the sense that it is not possible to indicate the reduction factor of one instance by the identification, even knowing the factor 0 instance. Furthermore, a series does not necessarily have to comprise all instances corresponding to each integer reduction factor.

FIG. 4 schematically shows a particular image having three instances. The instances are arbitrarily numbered, the image comprising an instance 2 formed by 4×4 frames (reduction factor 1), an instance 1 formed by a single frame (reduction factor 3), and an instance 3 formed by 8×8 frames (reduction factor 0). Thus, a 2×2 frame instance having a reduction factor of 2 is missing. Further, while each cell within each instance is identified by means of a frame number, but the frame numbers are arbitrarily assigned to each frame in an instance. In this context, exemplary frames A and B would be identified as follows:

Frame A: Instance 3, Frame number 14

Frame B: Instance 2, Frame number 5

This apparently random structure meets the DICOM standard, and therefore a well-designed DICOM viewer must be able to display such an image. Image information within the DICOM standard is stored in a dedicated system known as PACS (Picture Archiving and Communication System). Therefore, when requesting information to the PACS, a DICOM image viewer must request certain information from the PACS by means of frame number and instance.

Obviously, a conventional proprietary viewer designed to request cell information to the relevant database by identifying cells by means of (level, row, column) cannot display DICOM images where the frame image information is identified by means of (instance, frame number). Neither instances match levels nor frame numbers match rows and columns.

There is thus a need in the art to develop a solution allowing conventional viewers to display DICOM images.

### DESCRIPTION OF THE INVENTION

The present invention solves the above-disclosed drawback by means of a novel computer implemented method capable of “translating” a request from a proprietary high-resolution image viewer made in terms of level, row, and column into a DICOM valid request made in terms of DICOM instance and DICOM frame number. This method can be carried out by an interface module located between the proprietary image viewer and the DICOM database or PACS, as shown in FIG. 5. The interface module receives a request from the viewer in terms of level/row/column and “translates” it into a PACS compatible request in terms of instance/frame number. The answer from the PACS containing the image file of the relevant frame then sent to viewer via the interface module.

The present invention therefore allows for existing viewers designed to operate using level/row/column based cell identification to access images stored in the PACS according to the DICOM structure based on instance/frame number. This is advantageous in that health care services do not need to acquire a new DICOM compatible viewer.

A first aspect of the present invention therefore discloses a computer implemented method for accessing a DICOM WSI database or PACS from a high-resolution image viewer by means of an intermediate interface module. The high-resolution image viewer is configured for requesting cell image files from a database by identifying cell level, row, and column. On the other hand, the PACS stores a DICOM series comprising several DICOM instances which, in turn, comprise a plurality of DICOM frames. In this context, each DICOM frame in the PACS is identified by means of DICOM instance number and DICOM frame number. The method comprises mainly the following steps:
1) Receiving, from the high-resolution image viewer, a request for a cell image file identified by cell level, row and column.
2) Determining, in the intermediate interface module, a DICOM instance corresponding to said level.
3) Determining, in the intermediate interface module, a DICOM frame number corresponding to said row and column.
4) Requesting, to the PACS, the relevant frame image file identified by said DICOM instance and DICOM frame number.
5) Receiving, from the PACS, the relevant frame image file.
6) Sending, to the high-resolution image viewer, the relevant frame image file.

Now, in order to carry out steps 2 and 3, the interface module must have a first correspondence table establishing a correlation between levels and DICOM instances and a second correspondence table establishing a correlation between cell row and column and DICOM frame number. In this context, the term “correspondence table” must be interpreted to refer to any kind of data structure capable of storing the relevant correspondence information. The invention is not limited strictly to the use of a correspondence table.

Therefore, the method of the invention further comprises an initial process, carried out by the interface module, of building a first correspondence table between level and DICOM instance and a second correspondence table between row and column and DICOM frame number. Said initial process comprises the following steps:
01) Requesting to the PACS metadata from all the instances of the series.
02) Finding the size of each instance in the metadata received.
03) Finding the biggest size corresponding to the image size (level 0).
04) Calculating a reduction factor of each instance by means of the following equation:

$$Reduction factor = {log}_{2} \frac{image size}{instance size}$$

where the image size is the size of the largest instance received.

Therefore, the result of these calculations carried out in the interface module is a list with the reduction factors of each of the instances. Note that the largest instance of the image will have a reduction factor of 0. Further, note that, unlike in the case of levels, instances corresponding to every integer reduction factor are not necessarily present. In the example shown in FIG. 4, the result of this step is that instance 3 has a reduction factor of 0, instance 2 has a reduction factor of 1, and instance 1 has a reduction factor of 3.
05) Sorting the instances according to their reduction factors.

Following with the example shown in FIG. 4, the sorted instances are:
Instance 3 (reduction factor 0)
Instance 2 (reduction factor 1)
Instance 1 (reduction factor 3)

This step allows the interface module to find out whether there are instances missing. In the above example, instances corresponding to reduction factors 0, 1, and 3 are present. Therefore, an instance corresponding to a reduction factor of 2, i.e. a 2×2 instance, is missing. This is relevant because known proprietary viewers expect levels corresponding to every integer reduction factor to be present.

06) Building a correspondence table between each DICOM instance and a level having the same reduction factor.

The interface module builds a correspondence table between the DICOM instances and the levels. In connection with the example shown in FIGS. 3 and 4, the table could be as follows:

| Level | DICOM instance |
|---|---|
| 0 | Instance 3 |
| 1 | Instance 2 |
| 2 | Missing |
| 3 | Instance 1 |

07) Finding, in the metadata received, the frame number and the DICOM frame position for each frame. In this context, it is known that the DICOM frame position refers to the coordinates in pixels of the left upper corner of the frame.

Considering the example shown in FIGS. 3 and 4 for frames A and B:
Frame A: frame number 14, frame position (968 pixels, 556 pixels)
Frame B: frame number 5, frame position (456 pixels, 812 pixels)

08) Obtaining the lowest horizontal and vertical coordinates from all the DICOM frame positions.

Once the interface module has received the frame position for all frames, i.e. the left upper corner coordinates of all frames, the left edge and the upper edge of the instance must correspond respectively to the lowest horizontal coordinate and to the lowest vertical coordinate from all the frame positions.

09) Determining that said lowest horizontal and vertical coordinates as the horizontal and vertical offset.

In case the coordinates corresponding to the left and upper edges of the instance obtained in the above step are not zero, then the interface module derives that there is a certain offset corresponding to the value of said edge coordinates.

That is, in the example represented in FIG. 4, the lowest horizontal coordinates in instance 2 correspond to frame numbers 16, 1, 15 and 4. Let us suppose then that the horizontal coordinate corresponding to said frames is 200 pixels. Similarly, the lowest vertical coordinates in instance 2 corresponds to frame numbers 16, 13, 7 and 10. Let us suppose then that the vertical coordinate corresponding to said frames is 300 pixels.

Therefore, in this step the interface module determines the value of a horizontal offset and a vertical offset: the horizontal offset is 200 pixels, and the vertical offset is 300 pixels.

10) Calculating the row and column of each DICOM frame by means of the following equation:

$$Row = \frac{( vertical coordinate - vertical offset )}{frame height} - 1$$

$$Column = \frac{( horizontal coordinate - horizontal offset )}{frame width} - 1$$

where the vertical coordinate refers to the vertical component of the relevant frame position in pixels and the horizontal coordinate refers to the horizontal component of the relevant frame position in pixels.

In the example shown in FIG. 4, let us suppose that the frame height and width is 256 pixels, which is a frequently used value. The horizontal and vertical offsets calculated above are respectively 200 pixels and 300 pixels. And the frame position of exemplary frames A and B were respectively (968 pixels, 556 pixels) for frame A and (456 pixels, 812 pixels) for frame B.

Now, the row and column corresponding to frames A and B is obtained:

Frame A: Row=(556−300)/256+1=2 Column=(968−200)/256+1 =4

Frame B: Row=(812−300)/256+1=3 Column=(456−200)/256+1=2

11) Building a correspondence table between each DICOM frame number and the corresponding row and column.

The interface module builds a correspondence table between the DICOM frame numbers and the rows and columns of each cell. Following with the example shown in FIGS. 3 and 4, only the correspondence for frames A and B is calculated:

| | DICOM frame number | Row, column |
|---|---|---|
| Frame A | 14 | (2, 4) |
| Frame B | 5 | (3, 2) |

This process is carried out for all the frames in each instance. The final result are two correspondence tables, or generally speaking data structures, that allow for the interface module to immediately translate a petition from a conventional high-resolution image viewer made in terms of level, row and column into DICOM defined parameters such as DICOM instance and DICOM frame number. The interface module can then request the relevant information from the PACS and pass it back to the conventional image viewer, as schematically shown in FIG. 5.

While the method of the invention is particularly designed to be executed in a computer, the invention further encompasses a computer program comprising instructions capable of causing a computer to carry out the method of the invention. The program may be in the form of font code, object code, or an intermediate code between font code and object code, such as a partially compiled form. Generally speaking, the program may be in any computer readable form capable of causing a computer to carry out the method of the invention.

The computer program may be stored in a storing means. The storing means could be of any kind, such as e.g. a ROM, a CD-ROM, a semiconductor ROM, a hard disk, etc. Alternatively, the storing means could be an ASIC adapted to store and execute the method of the invention. In any case, generally speaking the storing means includes any kind of means capable of storing the list of instructions making up the computer program.

The computer program may be supported in a carrier. In this context, when the program is incorporated in a signal that can be transported by cable or by any other means, such as an electrical or optical signal, the carrier takes the form of said cable or other means.

A second aspect of the present invention is directed to an intermediate interface module for accessing a DICOM WSI or PACS from a high-resolution image viewer. As disclosed above, the PACS stores a DICOM series comprising several DICOM instances which, in turn, comprise a plurality of DICOM frames. In this context, each DICOM frame in the PACS is identified by means of DICOM instance number and DICOM frame number. On the other hand, the high-resolution image viewer is configured for requesting cell image files from a database by identifying level, row, and column of said cell. The interface module is configured for carrying out the following steps:
1) Receiving, from the high-resolution image viewer, a request for a cell image file identified by level, row and column of said cell.
2) Determining, in the intermediate interface module, a DICOM instance corresponding to said level.
3) Determining, in the intermediate interface module, a DICOM frame number corresponding to said row and column.
4) Requesting, to the PACS, the relevant frame image file identified by said DICOM instance and DICOM frame number.
5) Receiving, from PACS, the relevant frame image file.
6) Sending, to the high-resolution image viewer, the relevant frame image file.

Further, the interface module is configured for carrying out an initial process of building a first correspondence table between level and DICOM instance and a second correspondence table between row and column and DICOM frame number. Said initial process comprises the following steps:
01) Requesting to the PACS metadata from all the instances of the series.
02) Finding the size of each instance in the metadata received.
03) Finding the biggest size corresponding to the image size (level 0).
04) Calculating the reduction factor of each instance by means of the following equation:

$$Reduction factor = {log}_{2} \frac{image size}{instance size}$$

where the image size is the size of the largest instance received.
05) Sorting the instances according to their reduction factors.
06) Building a correspondence table between each DICOM instance and a level having the same reduction factor.
07) Finding, in the metadata received, the frame number and the DICOM frame position for each frame, where the DICOM frame position refers to the coordinates in pixels of the left upper corner of the frame.
08) Obtaining the lowest horizontal and vertical coordinates from all the DICOM frame positions.
09) Determining that said lowest horizontal and vertical coordinates as the horizontal and vertical offset.
10) Calculating the row and column of each DICOM frame by means of the following equations:

$$Row = \frac{( vertical coordinate - vertical offset )}{frame height} - 1$$

$$Column = \frac{( horizontal coordinate - horizontal offset )}{frame width} - 1$$

11) Building a correspondence table between each DICOM frame number and the corresponding row and column.

In this context, the term “module” must be interpreted widely, such that it does not refer only to a physical module such as a server, computer, or the like, but also to a software module that can be run in the same computer or server where the conventional viewer is run.

### BRIEF DESCRIPTION OF THE DRAWINGS

FIG. 1 shows an example of a pathologic anatomy digitized by means of a plurality of small size photographs.

FIGS. 2a and 2b schematically show the pyramidal structure used for visualizing the digitized images.

FIG. 3 schematically shows a pyramidal data structure employed by a known viewer operating in terms of cell level/row/column.

FIG. 4 schematically shows a pyramidal data structure defined by DICOM operating in terms of cell instance/frame number.

FIG. 5 shows a schematic view of an intermediate interface module according to the present invention.

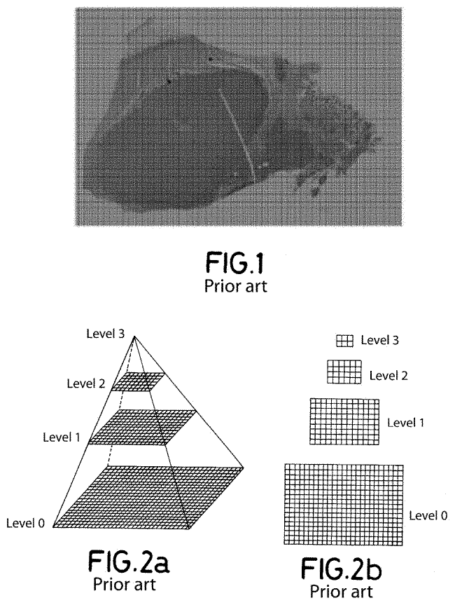

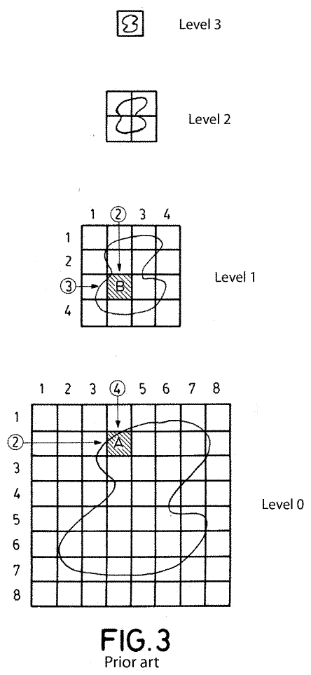

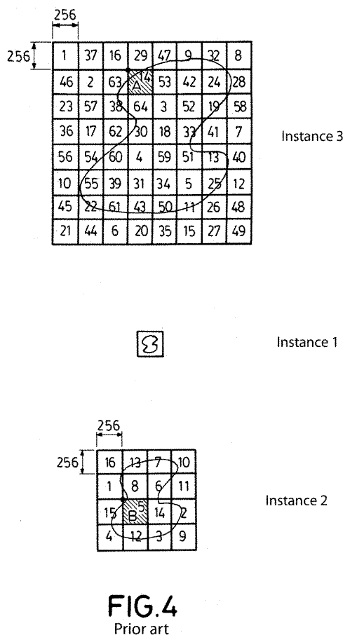

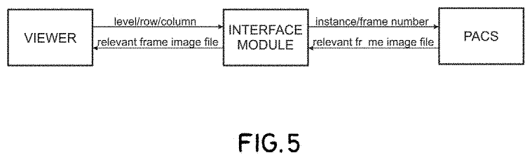

### PREFERRED EMBODIMENT OF THE INVENTION Examples illustrating the method of the invention is now disclosed in detail.

### EXAMPLES 1

First, the initial process for obtaining first and second correspondence tables respectively between level and instance and between row and column and frame number is carried out. This initial process comprises carrying out the following commands:
a) A C-GET command or a WADO-RD is sent from the interface module to the PACS requesting metadata about all instances of a series to be analyzed. The metadata corresponding to the instances are received, said metadata including the real size of the instances in tags “Total Image size: Column” y “Total Image size: Row”.

b) The reduction level of every instance is calculated by means of the formula

$$Reduction factor = {log}_{2} \frac{image size}{instance size}$$

The instances are sorted according to the corresponding reduction factors and a first corresponding table between instances and reduction factors is built.
c) A C-GET command or a WADO-RD is sent from the interface module to the PACS indicating an instance to get the metadata, including tags “Column position in pixels” y “Row position in pixels” if it weren't retrieved in a). The PACS returns a complete set of corresponding horizontal and vertical coordinates of the position of the left upper corner of each frame in the relevant instance.
d) In case the least value in “Column position in pixels” or “Row position in pixels” is greater than 1, said value is set as the corresponding horizontal and vertical offset.
e) The row and column of each frame is calculated using the following formulas:

$$Row = \frac{( vertical coordinate - vertical offset )}{frame height} - 1$$

$$Column = \frac{( horizontal coordinate - horizontal offset )}{frame width} - 1$$

f) A second correspondence table between frame number and cell row and column is built.

Alternatively, in case the PACS does not admit C-GET commands or the HTTP protocol is to be used, a WADO-RS command may be employed for gathering the same data and carrying out the same process.

Once the interface module has finished this initial process, access to the PACS from the conventional proprietary viewer based on cell level, row and column can start displaying DICOM images stored in the PACS. Each level/row/column based request from the viewer is “translated” into an instance/frame number request by the interface module by using the first and second correspondence tables. The PACS then returns the relevant frame image file to the interface module, which in turn passes said file to the viewer.

### EXAMPLE 2

In this case, the PACS configuration is altered to store certain metadata a client can request by means of a C-FIND (or QIDO) command. In that case, a C-FIND request with these metadata will be answered with this information and, in one request, all the information necessary for the algorithm will be obtained.

In this example, the process will comprise follow commands:
a) Modifying the PACS configuration such that the answer to a C-FIND/QIDO request contains the following metadata: “Total Image size: Column”, “Total Image size: Row”, “Per frame Sequence”, “Column position in pixels” y “Row position in pixels”.
b) Sending a C-FIND (or QIDO) request from the interface module to the PACS requesting all instances of a series to be analyzed. The answer will contain all the metadata needed.
c) Calculating the reduction level of every instance by means of the formula:

$$Reduction factor = {log}_{2} \frac{image size}{instance size}$$

d) Sorting the instances according to the corresponding reduction factors and building a first corresponding table between instances and reduction factors.
e) In case the lowest value in “Column position in pixels” or “Row position in pixels” is greater than 1, said value is set as the corresponding horizontal and vertical offset.
f) Calculating the row and column of each frame using the following formulas:

$$Row = \frac{( vertical coordinate - vertical offset )}{frame height} - 1$$

$$Column = \frac{( horizontal coordinate - horizontal offset )}{frame width} - 1$$

g) Building a second correspondence table between frame number and cell row and column.

1. A computer implemented method for accessing a Digital Imaging and Communications in Medicine (DICOM) Whole Slide Imaging (WSI) image database, or Picture Archiving and Communication System (PACS), from a high-resolution image viewer via an intermediate interface module,
wherein the PACS is configured to store a DICOM series comprising several DICOM instances which, in turn, comprise a plurality of DICOM frames, and wherein each DICOM frame in the PACS is identified by a DICOM instance number and a DICOM frame number, and
wherein the high-resolution image viewer is configured for requesting cell image files from a database by identifying a level, row, and column of said cell,
the method comprising:
receiving, from the high-resolution image viewer, a request for a cell image file identified by level, row and column of said cell;
determining, in the intermediate interface module, a DICOM instance corresponding to said level;
determining, in the intermediate interface module, a DICOM frame number corresponding to said row and column;
requesting, to the PACS, the relevant frame image file identified by said DICOM instance and DICOM frame number;
receiving, from the PACS, the relevant frame image file; and
sending, to the high-resolution image viewer, the relevant frame image wherein the method further comprises an initial process carried out by the interface module of building a first correspondence table between level and DICOM instance and a second correspondence table between cell row and column and DICOM frame number, said initial process comprising:
requesting, to the PACS, metadata from all the instances of the series.
finding the size of each instance in the metadata received;
finding the biggest size corresponding to the image size (level 0);
calculating a reduction factor of each instance by means of the following equation:
$$Reduction factor = {log}_{2} \frac{instance size}{image size}$$
wherein the image size is the size of the largest instance received;
sorting the instances according to their reduction factors;
building a correspondence table between each DICOM instance and a level having the same reduction factor;
finding, in the metadata received, the frame number and the DICOM frame position for each frame, wherein the DICOM frame position refers to the coordinates in pixels of the left upper corner of the frame;
obtaining the lowest horizontal and vertical coordinates from all the DICOM frame positions;
determining that said lowest horizontal and vertical coordinates as a horizontal and vertical offset;
calculating the row and column of each DICOM frame by means of the following equation:
$$Row = \frac{( vertical coordinate - vertical offset )}{frame height} - 1$$
$$Column = \frac{( horizontal coordinate - horizontal offset )}{frame width} - 1$$
and
building a correspondence table between each DICOM frame number and the corresponding row and column.

2. A non-transitory computer storage medium storing computer-executable instructions that, when executed by a processor, cause the processor to perfor the method according to claim 1.

3. (canceled)

4. (canceled)

5. An interface module for accessing a Digital Imaging and Communications in Medicine (DICOM) Whole Slide Imaging (WSI) image database or Picture Archiving and Communication System (PACS) from a high-resolution image viewer;
wherein the PACS stores a DICOM series comprising several DICOM instances which, in turn, comprise a plurality of DICOM frames, and wherein each DICOM frame in the PACS is identified a DICOM instance number and a DICOM frame number, and
wherein the high-resolution image viewer is configured for requesting cell image files from a database by identifying a level, row, and column of said cell,
wherein the interface module comprises a computer configured to:
receiveing, from the high-resolution image viewer, a request for a cell image file identified by level, row and column of said cell;
determine in the intermediate interface module, a DICOM instance corresponding to said level;
determine, in the intermediate interface module, a DICOM frame number corresponding to said row and column;
request, to the PACS, the relevant frame image file identified by said DICOM instance and DICOM frame number;
receive, from the PACS, the relevant frame image file; and
sending, to the high-resolution image viewer, the relevant frame image file; wherein the interface module is further configured to carry out an initial process of building a first correspondence table between level and DICOM instance and a second correspondence table between row and column and DICOM frame number, said initial process comprising:
requesting, to the PACS, the size of each instance;
calculating the reduction factor of each instance by means of the following equation:
$$Reduction factor = {log}_{2} \frac{instance size}{image size}$$
wherein the image size is the size of the largest instance received;
sorting the instances according to their reduction factors;
building a correspondence table between each DICOM instance and a level having the same reduction factor;
requesting, to the PACS, the DICOM frame number and the DICOM frame position for each DICOM frame, wherein the DICOM frame position refers to the coordinates in pixels of the left upper corner of the frame;
obtaining the lowest horizontal and vertical coordinates from all the DICOM frame positions;
determining that said lowest horizontal and vertical coordinates as the horizontal and vertical offset;
calculating the row and column of each DICOM frame by means of the following equation:
$$Row = \frac{( vertical coordinate - vertical offset )}{frame height} - 1$$
$$Column = \frac{( horizontal coordinate - horizontal offset )}{frame width} - 1$$
and
building a correspondence table between each DICOM frame number and the corresponding row and column.
